(19) United States
(12) Patent Application Publication
(10) Pub. No.: US 2023/0248284 A1
(43) Pub. Date: Aug. 10, 2023

(54) ENERGY MANAGEMENT MONITOR

(71) Applicant: Energy4Life Inc, Tampa, FL (US)

(72) Inventor: Harry MASSEY, Poole (GB)

(21) Appl. No.: 17/667,450

(22) Filed: Feb. 8, 2022

(51) Int. Cl. A61B 5/16; A61B 5/0205; A61B 5/024; A61B 5/11; A61B 5/1455; A61B 5/08; A61B 5/00

(52) U.S. Cl. CPC A61B 5/165; A61B 5/02055; A61B 5/02433; A61B 5/02438; A61B 5/1126; A61B 5/14551; A61B 5/0816; A61B 5/742; A61B 5/0008; A61B 5/7405; A61B 5/7455; A61B 2562/0219

(57) ABSTRACT

The present invention relates to an energy management monitor and to a system for detecting and monitoring emotions and an emotional level of a user. The monitor is adapted to be in direct contact with a user's skin. The monitor includes at least one sensor, a power supply, a memory which stores data obtained from the at least one sensor, and a wireless transmitter which is operative to transmit the data to a wireless receiver, upon receipt of an authorisation signal. The at least one sensor is a photoplethysmogram sensor which is operative to obtain data relating to: (i) a pulse duration (T), (ii) phase differences (δ) occurring between a forward pressure wave peak and a reflected pressure wave peak, and (iii) a goodness-of-fit correlation (β) between a waveform shape and a continuous probability function in order to determine an emotion (such as anxiety) level of the wearer.

### FIELD

The present invention relates to an energy management monitor and, more particularly but not exclusively, to a monitor and a system for detecting and monitoring emotions and an emotional level of a user.

### BACKGROUND OF INVENTION

Anxiety levels amongst the population is increasing significantly as a result of increasing pressures within day to day life. People are working longer hours, under greater stress, and due to increasing fatigue are failing to find time to look after their own well-being. Many people are unaware that they are continuing in an extended, or continuous, period of anxiety or stress. Anxiety and other emotions can be harmful to a person's health and energy levels.

There is therefore a need for a device which can efficiently monitor and detect emotions and to aid a user in taking steps to improve their well-being.

### SUMMARY OF INVENTION

According to a first aspect of the present invention, there is provided a monitor adapted to be in direct contact with a user's skin, the monitor includes at least one sensor, a power supply, a memory which stores data obtained from the at least one sensor, and a wireless transmitter which is operative to transmit the data to a wireless receiver, upon receipt of an authorisation signal, wherein the at least one sensor is a photoplethysmogram sensor which is operative to obtain data relating to: (i) a pulse duration (T), (ii) phase differences (δ) occurring between a forward pressure wave peak and a reflected pressure wave peak, and (iii) a goodness-of-fit correlation (β) between a waveform shape and a continuous probability function in order to determine an emotion level of the wearer.

According to a second aspect of the present invention, there is provided a monitor adapted to be in direct contact with a user's skin, the monitor includes at least one sensor, a power supply, a memory which stores data obtained from the at least one sensor, and a wireless transmitter which is operative to transmit the data to a wireless receiver, upon receipt of an authorisation signal, wherein the at least one sensor is a photoplethysmogram sensor which is operative to obtain data relating to: (i) a pulse duration (T), (ii) phase differences (δ) occurring between a forward pressure wave peak and a reflected pressure wave peak, and (iii) a goodness-of-fit correlation (β) between a waveform shape and a continuous probability function in order to determine a level of at least one of: impatience, anxiety, fear, frustration and grief, of the wearer.

According to a further aspect of the present invention, there is provided a system comprising a monitor as hereindescribed, and a mobile communication device, such as smartphone or tablet, in communication, for example wireless communication, with the monitor. The mobile communication device preferably operates in accordance with application specific software (APP) and has a display that presents an image corresponding to indicate the determined anxiety level or impatience level or level of fear or level of frustration level or level of grief of the wearer.

In the aforementioned embodiments data is transmitted from a memory via a wireless transmitter to a wireless receiver for example for processing remotely, such as by a smartphone. It is understood that a processor may be included in the monitor so as to enable on-board processing of data to be performed and an onboard display or feedback means may be included in some embodiments.

However, in a particularly preferred embodiment the monitor is small and lightweight and is configured to be in contact with a user's skin for at least 1 day, preferably a multiple day duration.

The monitor may comprise any suitable attachment means for securing the monitor in place in contact with a user's skin.

The monitor may be positioned adjacent any suitable area of the body of the user where pulse readings can be obtained. Suitable areas of the body include, for example, upper arm, wrist, temple, forehead, calf, ankle, or any combination thereof.

It is to be understood that the monitor may be secured in position by any suitable means, for example by a strap configured to extend around, in contact with, a suitable area of the body of the user.

The at least one sensor may be an accelerometer. The accelerometer is preferably operative to monitor a user's movement.

The at least one sensor is preferably an infrared (IR) sensor. The infrared (IR) sensor is preferably operative to measure a user's temperature.

The at least one sensor is preferably a pulse oximeter. The pulse oximeter is preferably operative to measure a user's blood oxygen level.

The respiratory rate is preferably derived from a number of peaks in an interbeat interval (IBI). The peaks in the IBI are preferably counted, during a predefined interval, and used to provide an indication of an anxiety level.

The pulse duration (T) is preferably classified as an intermediate pulse when its duration is less than 1.0 second or longer than 0.57 second in duration and may be used to provide an indication of an anxiety level. For example, the pulse duration (T) may be classified as an intermediate pulse when its duration is between 0.57 second and 1.0 second in duration and may be used to provide an indication of an anxiety level.

The intermediate pulse may be further classified into a first intermediate pulse and a second intermediate pulse. The first and second intermediate pulses preferably have a goodness-of-fit correlation (β) which is between 4.3-5.2. The combined duration of the first and second intermediate pulses is preferably less than 1.0 second and/or longer than 0.57 second, preferably less than 1.0 second and longer than 0.57 second.

The first intermediate pulse may have a goodness-of-fit correlation (β) which is between 4.3-5.2 and whose duration may be between 0.25-0.42 second. The second intermediate pulse may have a goodness-of-fit correlation (β) which is between 4.3-5.2 and whose duration may be between 0.42-0.60 second. The first and second intermediate pulses may be used to provide an indication of an anxiety level.

In one embodiment, a ratio of a sample of pulse measurements of first or second intermediate pulses/total of the sample of pulse measurements, is used to provide an indication of an anxiety level.

The data may be collected continuously or regularly, at predetermined intervals, over a time period. The predetermined intervals and/or time period may be adjusted by the user, for example in the firmware of the monitor and/or mobile communications device.

Table 1 below provides examples of other combinations of measured variables, referred to as characteristic factors, and how these are combined to provide an indication of some further emotion types.

In one embodiment, the monitor further comprises at least one light emitting diode (LED), or one or more groups of LEDs, each LED or group of LED being configured to provide a visible indication the status of the monitor, for example to provide a visible indication of one or more of: charging status, data transmittal status, data monitoring status, and/or battery level status.

The power supply preferably comprises an internal battery (e.g. an internal rechargeable battery). The power supply is preferably configured to power the monitor for at least one day, preferably for a multiple day period, for example for up to 1 week. The monitor preferably comprises a charging port.

The monitor may comprise conductive charging plates. The monitor may be configured to receive charge from a clip-on module comprising metal prongs configured to connect with the conductive plates on the monitor.

The monitor is preferably configured for wireless communication, for example Bluetooth® low energy communication wireless protocol, with the mobile communication device.

The memory is configured to store data obtained from the at least one sensor. When the monitor is not in communication with the mobile communication device (for example, when the mobile communication device is not nearby or switched off), the memory continues to store data from the at least one sensor and then when the mobile communication device is in communication with the monitor, the wireless transmitter is configured to allow all of the stored data obtained during the period of non-communication to be transmitted to the mobile communication device, either automatically or on request.

The monitor may comprise a housing configured to enclose, for example encompass, the at least one sensor, the power supply, the memory which stores data obtained from the at least one sensor, and a wireless transmitter to protect these components from exposure to the external environment, to increase durability of the monitor, and to prevent for example dirt and/or water ingress. The monitor, for example the housing, is preferably waterproof. The housing is preferably composed of a synthetic plastics material. The housing may for example be a welded plastics enclosure.

In one embodiment, at least a portion of the housing is transparent. In an alternative embodiment a housing may include display.

The display of the mobile communication device may be configured to present a calming image or a calming video. The display of the mobile communication device may be configured to present a calming image or a calming video on determination of a level of anxiety above a predetermined level of anxiety.

The mobile communication device may further comprise a speaker configured to present or play one or more images and/or soundtracks on determination of level of anxiety or a level of impatience (for example a level of anxiety or impatience greater than a predetermined level of anxiety or impatience) by the monitor.

In one embodiment, the mobile communication device is configured to present or play one or more calming image(s) and/or calming video(s) on determination of level of anxiety or a level of impatience (for example a level of anxiety or impatience greater than a predetermined level of anxiety or impatience) by the monitor.

The display of the mobile communication device may be configured to present a calming image or a calming video on determination of a level of anxiety above a predetermined level of anxiety. In one embodiment, the speaker is configured to present or play a meditation track. The use of a meditation track may help to reduce the levels of anxiety detected by the monitor and help bring the levels of anxiety towards a normalised level thereby reducing the associated negative effects of raised levels of anxiety on the user.

The soundtracks, images and/or videos may be selected by the user from a library of soundtracks, images and/or videos. In one embodiment, the library may comprise a plurality of different categories of soundtracks, images and/or videos, in which a particular category is selected to be presented if the monitor determines a predetermined level of anxiety or impatience.

Optionally a haptic feedback device outputs haptic feedback signals.

In one embodiment, the system further comprises a pulse emitting device configured to be worn around the wrist of a user. The pulse emitting device comprises an electromagnetic coil. The pulse emitting device is preferably configured to be communication with the mobile communications device. The pulse emitting device is preferably configured, on activation by a user via the mobile communications device, activated wirelessly by for example Bluetooth® connection, to generate pulse electromagnetic fields (PEMF) having a specific set of frequencies.

Frequencies are selected to have a therapeutic benefit to the user to reduce the associated negative effects of raised levels of anxiety on the user. The user may select the specific set of frequencies from a plurality of available options on the mobile communications device. The frequencies may be user specific and selected as a result of the user's data determined by the monitor.

The monitor and system of the present invention therefore provide an effective, personal energy management system (for example a personal anxiety and/or impatience management system). The monitor may include a haptic feedback means, such as pulse emitting device to aid stabilisation of emotions and to increase the energy levels of a user.

### BRIEF DESCRIPTION OF THE FIGURES

Embodiments of the present invention will now be described in more detail with reference to the accompanying Figures:

FIG. 1 is a schematic illustration of a flow chart for operation of the system according to one embodiment of the present invention; and

FIG. 2 is a graph of the anxiety levels detected by a monitor according to one embodiment of the present invention attached to the skin of a user.

### DETAILED DESCRIPTION

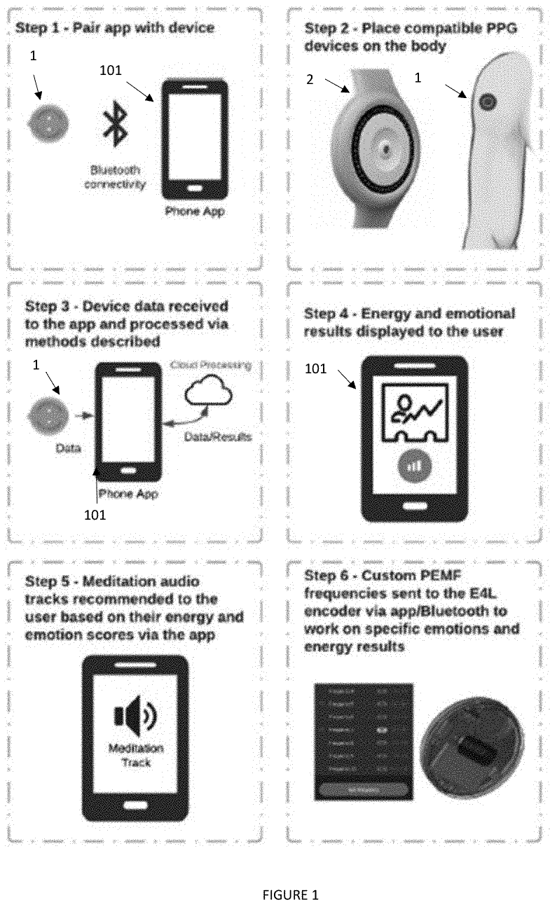

With reference to FIG. 1, the monitor 1 comprises at least one sensor, a power supply, a memory which stores data obtained from the at least one sensor, and a wireless transmitter which is operative to transmit the data to a wireless receiver, upon receipt of an authorisation signal. The at least one sensor is a photoplethysmogram sensor which is operative to obtain data relating to: (i) a pulse duration (T), (ii) phase differences (δ) occurring between a forward pressure wave peak and a reflected pressure wave peak, and (iii) a goodness-of-fit correlation (β) between a waveform shape and a continuous probability function in order to determine an anxiety level of the wearer.

By using a photoplethysmogram (PPG) sensor, the pulse waveform of a user can be visualised in software through the measurement of blood volume changes in tissue. Each emotional state of a user has been found to provide a different pulse waveform.

The at least one sensor includes an accelerometer operative to monitor a user's movement; an infrared (IR) sensor operative to measure a user's temperature; and a pulse oximeter operative to measure a user's blood oxygen level.

The monitor 1 further comprises light emitting diodes (LED) configured to provide a visible indication to the user of the status of the monitor 1, for example to provide a visible indication of one or more of: charging status, data transmittal status, data monitoring status, and/or battery level status.

The power supply comprises an internal rechargeable battery configured to power the monitor for at least one week. The monitor also comprises a charging port to enable one or more onboard batteries to be recharged.

The monitor comprises a housing configured to substantially encompass the sensors, the power supply, the memory which stores data obtained from the at least one sensor, and a wireless transmitter in order to protect these components from exposure to the external environment, to increase durability of the monitor, and to prevent for example dirt and/or water ingress.

The housing may comprise a synthetic plastics material and preferably comprises a transparent portion.

In the illustrated embodiment, the monitor 1 is paired, by Bluetooth® connectivity, to a mobile communications device 101 (for example a smartphone or tablet) prior to being positioned on a body of a user.

The monitor 1 is then placed in position on a wrist of a user. It is however to be understood that the monitor may be positioned adjacent any suitable area of the body of the user where pulse readings can be obtained. Suitable areas of the body include, for example, upper arm, wrist, temple, forehead, calf, ankle, or any combination thereof.

The monitor may comprise any suitable attachment means for securing the monitor in place in contact with a user's skin.

A pulse emitting device 2 is worn on the wrist of the user. The pulse emitting device 2 comprises an electromagnetic coil and is in communication with the mobile communications device 101.

In use, the sensors of the monitor 1 collect data which are stored within the memory of the monitor 1 and wirelessly transmitted to the mobile communications device 101.

Individual pulses can be classified into different emotional states of the user. Successful classification requires four parameters to be determined for each pulse: the pulse duration, the phase difference between the forward and reflected waves, and the ratio of the forward and reflected peaks, and a goodness-of-fit correlation. Each emotion of a user has been found to provide a specific waveform shape.

When the monitor 1 is not in communication with the mobile communication device 101, the memory continues to store data from the sensors and then when the mobile communication device 101 is in communication with the monitor 1, the wireless transmitter is configured to allow all of the stored data obtained during the period of non-communication to be transmitted to the mobile communication device 101, either automatically, at predetermined time intervals, or on request.

The results of the obtained data, and identified waveforms, are then provided to the user, on the mobile communication device 101, with lifestyle recommendations. The display of the mobile communication device 101 may provide a visual indication as to the anxiety level and/or impatience level and energy level of the user

The display of the mobile communication device 101 may for example, in response to the obtained data, present a calming image or a calming video on determination of a level of anxiety above a predetermined level of anxiety. It is to be understood that the display may suggest to the user that a calming image or video may be presented as a result of the determined level of anxiety of the user.

The speaker of the mobile communication device 101 may present or play one or more images and/or soundtracks on determination of level of anxiety or a level of impatience (for example a level of anxiety or impatience greater than a predetermined level of anxiety or impatience) by the monitor.

It is to be understood that the display may suggest to the user that a sound, video or soundtrack may be played as a result of the determined level of anxiety of the user.

The soundtracks, images and/or videos may be selected by the user from a library of soundtracks, images and/or videos.

By listening to and/or looking at the images, videos and/or soundtracks provided by the mobile communications device 101, the anxiety levels of a user may be reduced over a period of time towards a normalised level.

Table 1 below sets out some further combinations of sets of different characteristic factors and how these indicate a particular emotion type. Some examples of emotion types are: of: impatience, anxiety, fear, frustration and grief. However, it is understood that different emotion types or even sub-classes of emotion types of the wearer are now able to be detected and measured by selecting categories and combinations of the characteristic factors.

It is further understood that intelligent systems, which may include machine learning algorithms or other artificial intelligence (AI) devices may be used to improve sensitivity or reliability of emotion selection and measurement. Such as machine learning algorithms or other artificial intelligence (AI) devices may include further inputs from other devices or external or environmental data sources which are processed by a processor, such as a microprocessor. Other data sources, include for example, weather conditions including ambient temperature, location, time of day and total time that the wearer has been awake.

Examples of the characteristic factors are: L: pulse duration, δ: phase difference of peaks (time between systolic and diastolic peaks), R systolic/diastolic peak heights ratio and β: shape parameter, “goodness of fit”.

TABLE 1

| Emotions | Characteristic factors |
|---|---|
| Impatience | 0.57 < L < 1.0; 0.25 < δ/L < 0.42; R < 5.0 |
| | 0.57 < L < 1.0; δ/L < 0.17; β > 4.2 |
| Anxiety | 0.57 < L < 1.0; 0.42 < δ/L < 0.60; β < 5.3 |
| | 0.57 < L < 1.0; 0.25 < δ/L < 0.42; 4.3 < β < 5.3 |
| Fear | 0.57 < L < 1.0; 0.17 < δ/L < 0.25 |
| Frustration | 0.57 < L < 1.0; 0.25 < δ/L < 0.42; 3.3 < β < 4.3; |
| | R > 5.0 |
| | 0.57 < L < 1.0; δ/L < 0.17; 3.3 < β < 4.3 |
| Grief | L < 0.57 |
| | 0.57 < L < 1.0; δ/L < 0.17; β < 3.3 |

The pulse emitting device 2, on activation by a user via the mobile communications device (activated wirelessly by for example Bluetooth® connection) 101, generates pulse electromagnetic fields (PEMF) having a specific set of frequencies. The frequencies are selected to have a therapeutic benefit to the user to reduce the associated negative effects of raised levels of anxiety on the user. The frequencies are user specific and selected as a result of the user's data determined by the monitor.

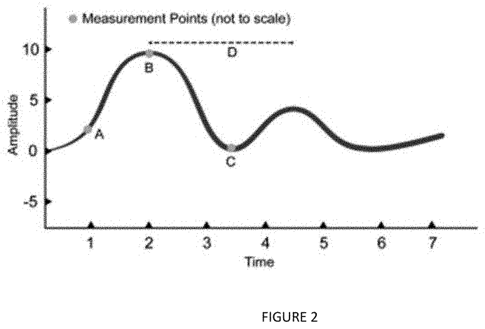

As shown in FIG. 2, the levels of anxiety of a user are measured, at regular intervals, using the monitor of the present invention over a predetermined time period. The determined levels of anxiety trigger an outcome from the pulse emitting device 2 (in the generation of PEMF) and/or from the mobile communications device 101 (in the generation of sounds, videos and/or images) in order to reduce the anxiety levels of a user efficiently and effectively.

The system of the present invention therefore, as well as detecting and measuring a particular emotion type, may be used to provide user specific recommendations for reducing particular levels of stress or anxiety or fear, for example by presenting one or more images, sounds and/or videos together with the generation of pulse electromagnetic fields (PEMF) or other types of haptic feedback. As a result of identified levels of anxiety or impatience of the user, these recommendations or feedback cues, may be displayed or played in order to reduce anxiety levels and to increase energy levels of the user.

It will be appreciated that the embodiments hereindescribed are examples only and that variation may be made to them, for example by detecting and measuring different variables relating to different moods (or energy levels) and combining and aggregating these variables in different ways.

1. A monitor adapted to be in direct contact with a user's skin, the monitor includes at least one sensor, a power supply, a memory which stores data obtained from the at least one sensor and a wireless transmitter which is operative to transmit the data to a wireless receiver, upon receipt of an authorisation signal, wherein the at least one sensor is a photoplethysmogram sensor which is operative to obtain data relating to: (i) a pulse duration (T), (ii) phase differences (δ) occurring between a forward pressure wave peak and a reflected pressure wave peak, and (iii) a goodness-of-fit correlation (β) between a waveform shape and a continuous probability function in order to determine an emotion level of the wearer.

2. The monitor according to claim 1 wherein the emotion is from the group of emotions comprising: impatience, anxiety, fear, frustration and grief, of the wearer.

3. The monitor according to claim 1, wherein the at least one sensor is an accelerometer and is operative to monitor a user's movement.

4. The monitor according to claim 1 wherein the at least one sensor is an infrared (IR) sensor and is operative to measure a user's temperature.

5. The monitor according to claim 1 wherein the at least one sensor is a pulse oximeter and is operative to measure a user's blood oxygen level.

6. The monitor according to claim 1 wherein a respiratory rate is derived from a number of peaks in an interbeat interval (IBI).

7. The monitor according to claim 6, wherein peaks in the IBI are counted, during a predefined interval and used to provide an indication of an anxiety level.

8. The monitor according to claim 1, wherein a pulse duration (T) is classified as an intermediate pulse when its duration is less than 1.0 second or longer than 0.57 second in duration and is used to provide an indication of an anxiety level.

9. The monitor according to claim 8 wherein the intermediate pulse is further classified into a first intermediate pulse, whose goodness-of-fit correlation (β) is between 4.3-5.2 and whose duration is between 0.25-0.42 second; and a second intermediate pulse, whose goodness-of-fit correlation (β) is between 4.3-5.2 and whose duration is between 0.42-0.60 second and is used to provide an indication of an anxiety level.

10. The monitor according to claim 8 wherein a ratio of a sample of pulse measurements of first or second intermediate pulses/total of the sample of pulse measurements, is used to provide an indication of an anxiety level.

11. The monitor according to claim 1 wherein a light emitting diode (LED) indicates a status of the monitor.

12. The monitor according to claim 1 includes a charging port.

13. The system includes a mobile communication device, such as smartphone, according to claim 1, which operates in accordance with application specific software (APP) and has a display that presents an image corresponding to indication of an anxiety level.

14. The system according to claim 13 wherein a display presents a calming image or a calming video.

15. The system according to claim 13 wherein a speaker plays a meditation track.

16. The system according to claim 13 wherein a haptic feedback device outputs haptic feedback signals.

17. The system according to claim 13 wherein an intelligent system includes a machine learning algorithms and/or an artificial intelligence (AI) device, is operative to improve sensitivity or reliability of emotion selection and measurement.

18. The system according to claim 13 wherein inputs are received by a processor from data sources from other external devices.
